(19) United States
(12) Patent Application Publication
(10) Pub. No.: US 2023/0285732 A1
(43) Pub. Date: Sep. 14, 2023

(54) Adjustable clamping-type ultrasonic medicine-applying massage device

(71) Applicant: Zhijun PENG, Foshan (CN)

(72) Inventor: Zhijun PENG, Foshan (CN)

(21) Appl. No.: 18/116,319

(22) Filed: Mar. 2, 2023

(30) Foreign Application Priority Data
Mar. 13, 2022 (CN) 202210244699.0

(51) Int. Cl. A61M 37/00; A61H 23/02

(52) U.S. Cl. CPC A61M 37/0092; A61H 23/0245; A61H 23/0254; A61M 2037/0007; A61H 2201/105; A61H 2201/0192; A61H 2201/1676; A61H 2201/0119; A61H 2201/1692

(57) ABSTRACT

An adjustable clamping-type ultrasonic medicine-applying massage device, including a left clamping arm, a right clamping arm, an intermediate hinge adjustment and ultrasonic medicine-applying massage heads; an end of the left clamping arm is in an articulated connection with an end of the right clamping arm through the intermediate hinge adjustment mechanism; an operating space is formed in an area where the left clamping arm and the right clamping arm face to each other; another end of the left clamping arm and another end of the right clamping arm are each provided with a ultrasonic medicine-applying massage head; the ultrasonic medicine-applying massage head of the left clamping arm and the ultrasonic medicine-applying massage head of the right clamping arm face towards each other.

### FIELD OF THE INVENTION

The present invention relates to the field of ultrasonic massage devices, and more particularly a therapeutic device with ultrasonic medicine-applying massage functions.

### BACKGROUND OF THE INVENTION

A therapeutic massage device in the prior art applied to the neck, waist, legs or arms of a human body to perform vibration massage contains two major problems: first, the clamping of the therapeutic massage device is achieved solely by the elasticity of the material of the device itself, thus having a poorer clamping stability; as the therapeutic massage device performs massage by high-frequency vibration, the device is prone to fall off if it has a poor clamping stability, which not only affects the massage effects of the device, but also the user experience; second, a structure of the therapeutic massage device is not capable of being adjusted, which does not allow a user to adjust the device optimally for the greatest comfort to wear according to a size of one's body parts, thus significantly affecting the user experience and massage effects.

### BRIEF SUMMARY OF THE INVENTION

It is an object of the present invention to overcome the above problems of the prior art, by providing an adjustable clamping-type ultrasonic medicine-applying massage device which allow a user to adjust a clamping angle according one's needs, with easy operation, stable clamping, comfortable wearing, and good massage effects.

The present invention provides the following technical solutions: an adjustable clamping-type ultrasonic medicine-applying massage device, comprising a left clamping arm, a right clamping arm, an intermediate hinge adjustment mechanism, and ultrasonic medicine-applying massage heads; an end of the left clamping arm is in an articulated connection with an end of the right clamping arm through the intermediate hinge adjustment mechanism; an operating space is formed in an area where the left clamping arm and the right clamping arm face to each other; another end of the left clamping arm and another end of the right clamping arm are each provided with a corresponding ultrasonic medicine-applying massage head; the ultrasonic medicine-applying massage head of the left clamping arm and the ultrasonic medicine-applying massage head of the right clamping arm face towards each other.

Furthermore, each of the left clamping arm and the right clamping arm comprises a front supporting arm, a rear supporting arm and a first lock nut; the front supporting arm is longitudinally movably fitted with a front end of the rear supporting arm; the first lock nut is provided at a front end opening of the rear supporting arm to fix the front supporting arm.

Furthermore, a front end of the front supporting arm is provided with a horizontal adjustment mechanism; the horizontal adjustment mechanism is connected with a corresponding ultrasonic medicine-applying massage head.

Furthermore, the intermediate hinge adjustment mechanism comprises a left hinge portion, a right hinge portion, a lock button, a spring, an annular toothed ring and stoppers; the left hinge portion is connected to the rear supporting arm of the left clamping arm; the right hinge portion is connected to the rear supporting arm of the right clamping arm; the left hinge portion and the right hinge portion are hinged together; the stoppers are provided on the right hinge portion; the left hinge portion is provided with a button base; the spring and the lock button are sequentially installed inside the button base; the annular toothed ring is provided on the lock button; the annular toothed ring and the stoppers are meshed for position locking under effect of the spring.

By adopting the aforementioned technical solutions, the beneficial effects of the present invention are as follows: the present invention provides an adjustable clamping-type ultrasonic medicine-applying massage device, comprising a left clamping arm, a right clamping arm, an intermediate hinge adjustment and ultrasonic medicine-applying massage heads, which allows a user to adjust a size of the clamping space between the left clamping arm and the right clamping arm according to one's needs; after adjustment, the intermediate hinge adjustment mechanism performs position locking and ensures clamping stability of the present invention, which provides advantages such as easy operation, stable clamping, comfortable wearing and good massage effects; when the ultrasonic medicine-applying massage heads are operating, they perform high-frequency ultrasonic massage while applying gaseous medication to a human body; high-frequency ultrasonic massage facilitates blood flow in the body and such increased circulation in the body will increase body temperature and help the pores on the skin to open to receive the gaseous medication, which allows medical ingredients of the gaseous medication to be quickly and effectively absorbed by the body, thereby improving the therapeutic effects and greatly reducing the duration of treatments.

### BRIEF DESCRIPTION OF THE DRAWINGS

FIG. 1 shows a structural view of the present invention;

FIG. 2 shows the left clamping arm, the right clamping arm and the intermediate hinge adjustment mechanism of the present invention in an exploded structure;

FIG. 3 shows a bottom perspective view of the right hinge portion of the present invention;

FIG. 4 shows a structural view of the ultrasonic medicine-applying massage head of the present invention.

### DETAILED DESCRIPTION OF THE INVENTION

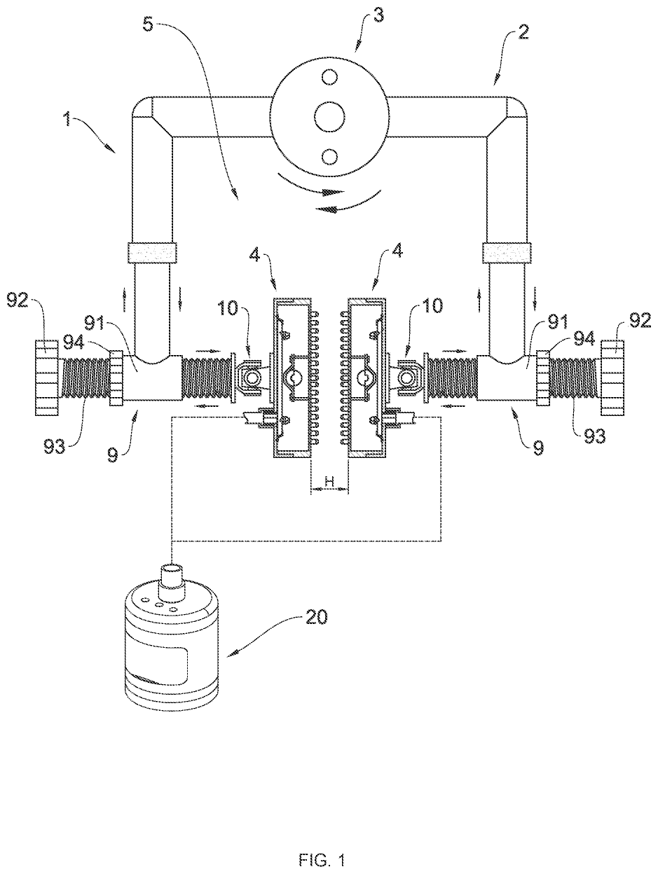

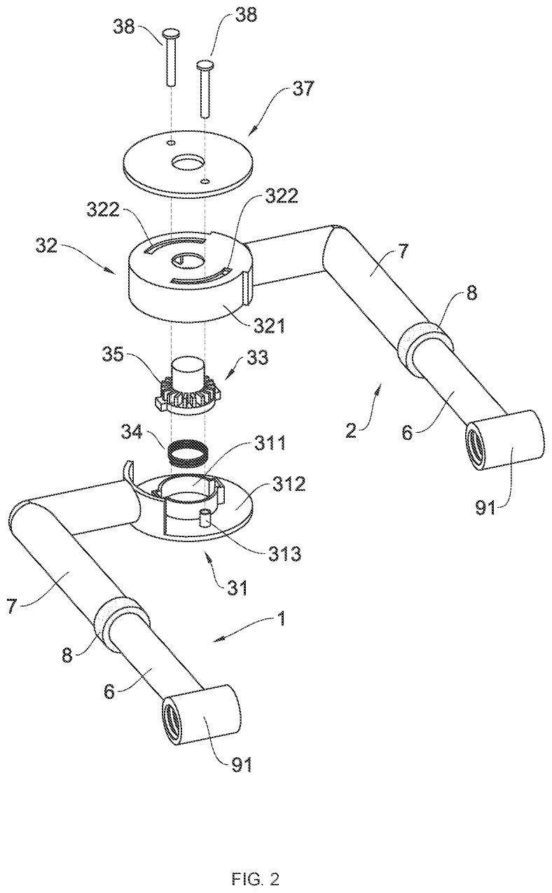

As illustrated in FIGS. 1-2, the present invention provides an adjustable clamping-type ultrasonic medicine-applying massage device, comprising a left clamping arm 1, a right clamping arm 2, an intermediate hinge adjustment mechanism 3, and ultrasonic medicine-applying massage heads 4; an end of the left clamping arm 1 is in an articulated connection with an end of the right clamping arm 2 through the intermediate hinge adjustment mechanism 3; an operating space 5 is formed in an area where the left clamping arm 1 and the right clamping arm 2 face to each other; another end of the left clamping arm 1 and another end of the right clamping arm 2 are each provided with a corresponding ultrasonic medicine-applying massage head 4; the ultrasonic medicine-applying massage head 4 of the left clamping arm 1 and the ultrasonic medicine-applying massage head 4 of the right clamping arm 2 face towards each other; the intermediate hinge adjustment mechanism 3 of the present invention enables an adjustment of a size of the operating space 5, thus being adaptive to users with different body shapes, and allowing users to conveniently adjust a clamping force of the left clamping arm 1 and the right clamping arm 2 on a body part according to their needs; after an adjustment, the intermediate hinge adjustment mechanism 3 performs position locking and ensures a clamping stability of the present invention, which provides advantages such as easy operation, stable clamping, comfortable wearing and good massage effects, and will not fall off due to loose clamping; when in use, first activate the intermediate hinge adjustment mechanism 3, rotate the left clamping arm 1 and the right clamping arm 2 to move to a suitable position, clamp a body part of a user, such as an arm or a waist, with an inner side surface of each ultrasonic medicine-applying massage head 4; after adjusting position of the left clamping arm 1 and the right clamping arm 2 to a suitable position which is capable of clamping stably, the user can turn on the medicine-applying massage heads 4 to perform ultrasonic vibration massage; gaseous medication can be supplied to the medicine-applying massage heads 4 through an external gaseous medication supplying device 20, so as to perform massage and medicine-applying at the same time;

As illustrated in FIG. 2, the left clamping arm 1 and the right clamping arm 2 each comprises a front supporting arm 6, a rear supporting arm 7 and a first lock nut 8; each front supporting arm 6 is longitudinally movably fitted with a front end of a corresponding rear supporting arm 7; each first lock nut 8 is provided at a front end opening of a corresponding rear supporting arm 7 to fix a corresponding front supporting arm 6; when a length of each front supporting arm 6 extending out of a corresponding rear supporting arm 7 needs to be adjusted, first loosen a corresponding first lock nut 8, and then each front supporting arm 6 can be pulled out from or inserted into the corresponding rear supporting arm 7; after adjustment, tighten each corresponding first lock nut 8; the above design allows a longitudinal length of the left clamping arm 1 and the right clamping arm 2 to be adjusted; therefore, when the present invention is not in use, it can be adjusted to a smaller size, which is convenient to be stored or packed for transportation; when the present invention is in use, it can be adjusted to different lengths and clamping angles and provides better adaptability.

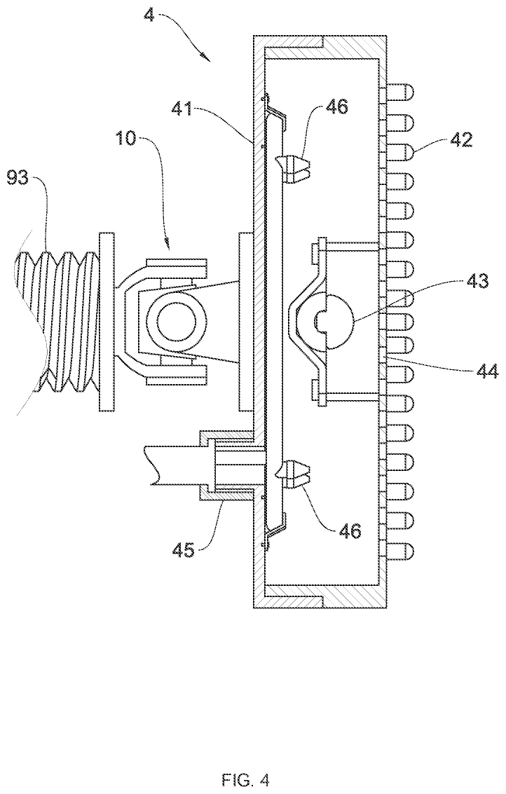

To achieve an adjustment of the ultrasonic medicine-applying massage heads 4 in a horizontal direction, as illustrated in FIG. 1, a front end of each front supporting arm 6 is further provided with a horizontal adjustment mechanism 9; each ultrasonic medicine-applying massage head 4 is connected to a corresponding horizontal adjustment mechanism 9; a size of a clamping gap, namely a clamping position H (as illustrated in FIG. 1) can be easily adjusted through the horizontal adjustment mechanisms 9, which allow a user to adjust a suitable clamping force and provide a more comfortable operating process; furthermore, to provide the horizontal adjustment mechanisms 9 with a simple structure, convenient adjustment, easy operation and low cost, as illustrated in FIG. 1, each horizontal adjustment mechanism 9 comprises a bolt base 91 connected to the front end of a corresponding front supporting arm 6, a bolt 93, a handle 92 connected to the bolt 93, and a second lock nut 94; the bolt 93 is connected to the bolt base 91; the second lock nut 94 is connected to the bolt 93; a corresponding ultrasonic medicine-applying massage head 4 is connected to a front end of the bolt 93; furthermore, to enable the corresponding ultrasonic medicine-applying massage head 4 to form a more suitable and comfortable clamping angle, as illustrated in FIG. 1 and FIG. 4, a universal joint 10 is provided between the corresponding ultrasonic medicine-applying massage head 4 and the front end of the bolt 93; the universal joint 10 can be a cross type universal joint or a ball type universal joint.

To provide the ultrasonic medicine-applying massage heads 4 with advantages such as simple structures, easy operation, high reliability and good durability, as illustrated in FIG. 4, each ultrasonic medicine-applying massage head 4 comprises an outer shell 41, a plurality of raised massaging bumps 42, an ultrasonic vibration component 43, a plurality of medicine spraying holes 44, and a tube connecting port 45; the ultrasonic vibration component 43 is provided inside the outer shell 41; the raised massaging bumps 42 are provided on a front surface of the outer shell 41; the medicine spraying holes 44 are provided on spaces between the raised massaging bumps 42; the tube connecting port 45 is provided on a rear surface or a side surface of the outer shell 41; furthermore, to enable the present invention to spray gaseous medication evenly with a certain degree of pressure, as illustrated in FIG. 4, a plurality of spray nozzles 46 facing the medicine spraying holes 44 are provided inside the outer shell 41; a rear end of each spray nozzle 46 is connected to the tube connecting port 45; another end of the tube connecting port 45 is connected to the gaseous medication supplying device 20 through a tube.

Furthermore, to provide the present invention with a better ultrasonic effect, the ultrasonic vibration component 43 is an ultrasonic transducer of 1 MHz or above; or, each ultrasonic vibration component 43 is an ultrasonic vibration motor with 10,000 revolutions per minute or above.

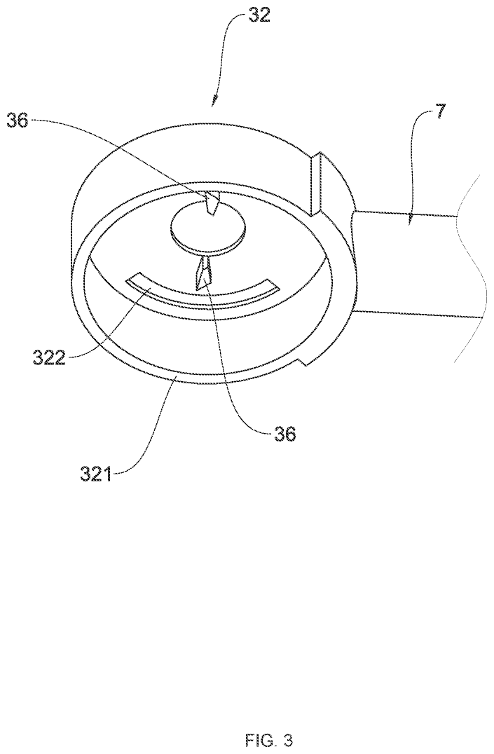

To provide the intermediate hinge adjustment mechanism 3 of the present invention with advantages such as easy operation, simple structure, high reliability and good durability, as illustrated in FIG. 2 and FIG. 3, the intermediate hinge adjustment mechanism 3 comprises a left hinge portion 31, a right hinge portion 32, a lock button 33, a spring 34, an annular toothed ring 35 and stoppers 36; the left hinge portion 31 is connected to the rear supporting arm 7 of the left clamping arm 1; the right hinge portion 32 is connected to the rear supporting arm 7 of the right clamping arm 2; the left hinge portion 31 and the right hinge portion 32 are hinged together; the stoppers 36 are provided on the right hinge portion 32; the left hinge portion 31 is provided with a button base 311; the spring 34 and the lock button 33 are sequentially installed inside the button base 311; the annular toothed ring 35 is provided on the lock button 33; the annular toothed ring 35 and the stoppers 36 are meshed for position locking under effect of the spring 34; in order to rotate the left clamping arm 1 and the right clamping arm 2 so as to adjust their positions, press the lock button 33 to overcome the resilient force of the spring 34, so that the annular toothed ring 35 on the lock button 33 disengage from the stoppers 36 on the right hinge portion 32; accordingly, the left hinge portion 31 and the right hinge portion 32 are rotatable with respect to each other so that their positions can be adjusted. After the left clamping arm 1 and the right clamping arm 2 have been adjusted to suitable positions, release the lock button 33 such that the resilient force of the spring 34 resets the lock button 33; accordingly, the annular toothed ring 35 is again meshed with the stoppers 36, and the left clamping arm 1 and the right clamping arm 2 are then locked in their adjusted positions. Furthermore, to improve an assembling stability of the intermediate hinge adjustment mechanism 3, as illustrated in FIG. 2, the intermediate hinge adjustment mechanism 3 further comprises a fixing piece 37 and screws 38; the left hinge portion 31 is arranged as a male hinge structure 312; the right hinge portion 32 is arranged as a female hinge structure 321; the male hinge structure 312 and the female hinge structure 321 are fitted with each other; the male hinge structure 312 is further provided with fixing screw holes 313; the female hinge structure 321 is further provided with curved holes 322; the fixing plate 37 is provided on a top surface of the female hinge structure 321; each screw 38 sequentially passes through the fixing plate 37 and a corresponding curved hole 322, and is fixed with a corresponding fixing screw hole 313, so that the left hinge portion 31 and the right hinge portion 32 are rotably fitted with each other.

When implementing the solutions of the present invention, an intelligent controlling IC chip and circuit board can be arranged inside the ultrasonic medicine-applying massage heads 4 or the gaseous medication supplying device 20, thereby controlling operation of the ultrasonic medicine-applying massage heads 4 or the gaseous medication supplying device 20 through the intelligent controlling IC chip and circuit board; furthermore, the intelligent controlling IC chip and circuit board can be provided with a Bluetooth® communication module or a WIFI® module operated with a corresponding controlling application software, so as to control the operation of the present invention with smart phones or tablets.

What is claimed is:

1. An adjustable clamping-type ultrasonic medicine-applying massage device, comprising a left clamping arm, a right clamping arm, an intermediate hinge adjustment mechanism, and ultrasonic medicine-applying massage heads; an end of the left clamping arm is in an articulated connection with an end of the right clamping arm through the intermediate hinge adjustment mechanism; an operating space is formed in an area where the left clamping arm and the right clamping arm face to each other; another end of the left clamping arm and another end of the right clamping arm are each provided with a corresponding ultrasonic medicine-applying massage head; the ultrasonic medicine-applying massage head of the left clamping arm and the ultrasonic medicine-applying massage head of the right clamping arm face towards each other.

2. The adjustable clamping-type ultrasonic medicine-applying massage device of claim 1, wherein each of the left clamping arm and the right clamping arm comprises a front supporting arm, a rear supporting arm and a first lock nut; the front supporting arm is longitudinally movably fitted with a front end of the rear supporting arm; the first lock nut is provided at a front end opening of the rear supporting arm to fix the front supporting arm.

3. The adjustable clamping-type ultrasonic medicine-applying massage device of claim 2, wherein a front end of the front supporting arm is provided with a horizontal adjustment mechanism; the horizontal adjustment mechanism is connected with a corresponding ultrasonic medicine-applying massage head.

4. The adjustable clamping-type ultrasonic medicine-applying massage device of claim 3, wherein each horizontal adjustment mechanism comprises a bolt base connected to the front end of a corresponding front supporting arm, a bolt, a handle connected to the bolt, and a second lock nut; the bolt is connected to the bolt base; the second lock nut is connected to the bolt; a corresponding ultrasonic medicine-applying massage head is connected to a front end of the bolt.

5. The adjustable clamping-type ultrasonic medicine-applying massage device of claim 4, wherein a universal joint is provided between the corresponding ultrasonic medicine-applying massage head and the front end of the bolt.

6. The adjustable clamping-type ultrasonic medicine-applying massage device of claim 5, wherein each ultrasonic medicine-applying massage head comprises an outer shell, a plurality of raised massaging bumps, an ultrasonic vibration component, a plurality of medicine spraying holes, and a tube connecting port; the ultrasonic vibration component is provided inside the outer shell; the raised massaging bumps are provided on a front surface of the outer shell; the medicine spraying holes are provided on spaces between the raised massaging bumps; the tube connecting port is provided on a rear surface or a side surface of the outer shell.

7. The adjustable clamping-type ultrasonic medicine-applying massage device of claim 6, wherein a plurality of spray nozzles facing the medicine spraying holes are provided inside the outer shell; a rear end of each spray nozzle is connected to the tube connecting port.

8. The adjustable clamping-type ultrasonic medicine-applying massage device of claim 6, wherein the ultrasonic vibration component is an ultrasonic transducer of 1 MHz or above; or, the ultrasonic vibration component is an ultrasonic vibration motor with 10,000 revolutions per minute or above.

9. The adjustable clamping-type ultrasonic medicine-applying massage device of claim 2, wherein the intermediate hinge adjustment mechanism comprises a left hinge portion, a right hinge portion, a lock button, a spring, an annular toothed ring and stoppers; the left hinge portion is connected to the rear supporting arm of the left clamping arm; the right hinge portion is connected to the rear supporting arm of the right clamping arm; the left hinge portion and the right hinge portion are hinged together; the stoppers are provided on the right hinge portion; the left hinge portion is provided with a button base; the spring and the lock button are sequentially installed inside the button base; the annular toothed ring is provided on the lock button; the annular toothed ring and the stoppers are meshed under effect of the spring to lock a position of the left clamping arm relative to a position of the right clamping arm.

10. The adjustable clamping-type ultrasonic medicine-applying massage device of claim 9, wherein the intermediate hinge adjustment mechanism further comprises a fixing piece and screws; the left hinge portion is arranged as a male hinge structure; the right hinge portion is arranged as a female hinge structure; the male hinge structure and the female hinge structure are fitted with each other; the male hinge structure is further provided with fixing screw holes; the female hinge structure is further provided with curved holes; the fixing plate is provided on a top surface of the female hinge structure; each screw sequentially passes through the fixing plate and a corresponding curved hole, and is fixed with a corresponding fixing screw hole, so that the left hinge portion and the right hinge portion are rotably fitted with each other.
